(19) United States
(12) Patent Application Publication
(10) Pub. No.: US 2026/0194458 A1
(43) Pub. Date: Jul. 9, 2026

(54) GAS GENERATOR WITH HIERARCHICAL SOURCES AND GETTERS

(71) Applicant: Vapor Cell Technologies LLC, Boulder, CO (US)

(72) Inventors: Steven Edward Bopp, Boulder, CO (US); Douglas Bopp, Boulder, CO (US)

(21) Appl. No.: 19/444,020

(22) Filed: Jan. 8, 2026

Related U.S. Application Data
(60) Provisional application No. 63/743,161, filed on Jan. 8, 2025.

(51) Int. Cl. G01N 21/31; G01N 33/00

(52) U.S. Cl. CPC G01N 21/31; G01N 33/0027; G01N 2201/0227

(57) ABSTRACT

A gas generator includes (i) a plurality of sources composed of a first material, the plurality of sources including a first source having a first source area and a second source having a second source area less than the first source area, (ii) a plurality of getters composed of a second material different from the first material, the plurality of getters including a first getter having a first getter area and a second getter having a second getter area less than the first getter area, and (iii) a substrate on which the plurality of sources and the plurality of getters are mounted such that none of the plurality of sources directly touch each other, none of the plurality of getters directly touch each other, and none of the plurality of sources directly touch the plurality of getters.

### RELATED APPLICATIONS

This application claims priority to U.S. Provisional Ser. No. 63/743,161, filed Jan. 8, 2025, which is incorporated herein by reference in its entirety.

### BACKGROUND

Vapor cells used for atomic sensing and timing applications benefit from precise control of gas quantity and purity to ensure stable and accurate performance.

### SUMMARY

The present embodiments include systems and methods for precise control of gas generation and removal within microfabricated, hermetically sealed containers, such as vapor cells used in atomic sensing and timing applications. In embodiments, a gas generator includes a plurality of sources composed of a first material and a plurality of getters composed of a second material different from the first material. Each source and getter is patterned into discrete elements of varying size such that, upon activating a discrete element, the quantity of gas released or removed is based on the physical dimensions of the respective element. The sources and getters are mounted on a substrate in a configuration that ensures that the discrete elements are thermally isolated from each other, thereby preventing the activation of one source or getter from inadvertently activating another.

The gas generator enables fine-tuned regulation of gas pressure and purity within a vapor cell or a vacuum cell by sequentially activating sources and getters of different sizes, allowing incremental adjustments of the gas toward a target condition within the cell. A precursor material may be subdivided into a hierarchy of different sized sources or getters, where each source, upon activation, generates a predictable quantity of gas, and where each getter, upon activation, removes a predictable quantity of one or mores species of gas. This subdivision may be achieved using techniques that progressively partition the material into smaller, independently activatable elements, enabling the creation of spatially addressable structures that provide precise control over the amount of gas introduced or absorbed.

In some embodiments, the system incorporates a feedback mechanism configured to monitor the species present within the hermetically sealed container (e.g., a vacuum cell or a vapor cell) and dynamically adjust activation sequences. This feedback control may employ spectroscopic interrogation or other sensing techniques to determine gas pressure, purity, or composition, and may automatically trigger the subsequent activation of selected sources or getters to maintain or reach a target condition. The combination of hierarchical subdivision and feedback-driven activation enables adaptive trimming of gas quantities, providing consistent and repeatable regulation of gas conditions even when operational requirements vary.

In some of the embodiments, the sources and the getters lay on top of pedestals, which in turn lay on top of the substrate. These pedestals improve thermal isolation between the activatable elements and may promote uniform heat distribution within an element during activation. Pedestals may include single or multi-layer configurations with tailored thermal properties to prevent heat diffusion between adjacent sources and getters.

The sources and getters may be fabricated from various precursor materials suitable for generating or removing specific gases, and may be patterned using techniques such as laser trimming, micromachining, or other precision fabrication methods.

Advantageously, the systems and methods described herein enable modular, scalable gas management strategies that support miniaturized, chip-scale systems and next-generation atomic clocks, laser stabilization platforms, and other precision devices requiring robust and repeatable gas control.

### BRIEF DESCRIPTION OF THE FIGURES

FIG. 1 shows a top view of a gas generator, in accordance with some of the present embodiments.

FIG. 2 illustrates one example of a method for generating a gas within a vapor cell or a vacuum cell by sequentially activating the gas generator shown in FIG. 1.

FIG. 3A shows a top view of a gas generator.

FIG. 3B shows a top view of a gas generator that is similar to the gas generator of FIG. 3A.

FIG. 4A shows a side view of a patterned precursor material (i.e., a plurality of sources or a plurality of getters), in accordance with some of the present embodiments.

FIG. 4B shows a side view of a pedestaled precursor structure, in accordance with some of the present embodiments.

FIG. 4C illustrates a side view of a pedestaled precursor heterostructure, in accordance with the present embodiments.

### DETAILED DESCRIPTION

A vapor cell is a hermetically sealed container that confines a gas in an isolated environment for spectroscopic interrogation of an atomic or molecular species. The gas is ideally composed of only an atomic or molecular species (e.g., an isotope of an atomic element) with no other contaminant species present. Typically, one or more walls of the vapor cell is transparent so that the species can be spectroscopically measured (e.g., with a laser located outside of the vapor cell). Thus, the vapor cell enables high-precision spectroscopy that improves the performance of atomic and molecular-based sensing and timing. Systems like atomic clocks and laser-stabilization platforms, which rely on vapor cells, play a major role in supporting modern infrastructure including global navigation and telecommunication networks. The choice of gas (i.e., the atomic or molecular species) is pivotal in enabling these high-precision spectroscopic measurements.

The gas species to be used within a vapor cell depends on the intended application and the intrinsic properties of the atomic or molecular species. Alkali metals, such as rubidium and cesium, are commonly used in atomic clocks due to their well-known and stable hyperfine transitions, making them particularly suited for precise timekeeping. Acetylene, on the other hand, has a distinct absorption line at 1.5 μm, making it advantageous for stabilizing and calibrating lasers in fiber-optic communication systems. These properties enable vapor cells to drive advancements in industries ranging from GPS systems to high-speed internet.

Systems incorporating vapor cells, like chip-scale atomic clocks (CSACs), require precise control over the quantity and purity of the gas to optimize performance. An insufficient amount of gas produces weak signals that reduces clock stability, while excess gas leads to collisional broadening that degrades measurement accuracy (and also reduces clock stability). Prior methods, such as heating solid-state rubidium precursors or dispensing iodine vapor by heating a solid sample of iodine inside the cell, often introduce contaminants or lack the fine control needed to regulate the pressure (i.e., the number of gas particles that interact with a laser beam propagating through the cell). Once sealed, achieving a pure and precise amount of gas becomes increasingly complex.

One common method to generate a gas in a hermetically sealed vapor cell or vacuum cell is to release the gas by heating an alkali metal dispenser (AMD). An AMD relies on the thermal decomposition of solid-state precursors, precipitating the release of alkali vapors. However, concurrent chemical reactions and degassing of the precursors produces unwanted byproducts, necessitating strategies to remove contamination. AMDs typically incorporate getter materials such that the precursor material simultaneously releases gas and chemically sequesters contaminants. For example, an AMD designed to release rubidium gas might use a rubidium-zirconium compound. In this setup, zirconium helps remove contaminants like molecular hydrogen that form during the rubidium gas release, which improves the purity of the rubidium sample in the vapor cell.

Despite its effectiveness, this process presents challenges. AMD reactions proceed exclusively as a function of the heat input, which is difficult to control with precision and consistency, especially within the hermetic environment of a vapor cell or vacuum cell. Heat tends to diffuse into surrounding materials, leading to an uneven distribution across the AMD. This variability makes it challenging to regulate the exact amount of gas produced, affecting precision and repeatability. These limitations highlight the need for more precise, consistent, and adaptable gas delivery systems and methods.

The present embodiments include systems and methods that subdivide one or more post-deposition precursor materials into physically discrete, customizable elements. Each element is configured to, upon activation, generate or remove a predetermined amount of gas of an atomic or molecular species. The physically discrete elements may be formed from one or more precursor materials and may be selectively and independently activated to function as gas sources or getters.

When a source element is activated, it introduces a corresponding species into a vapor cell or vacuum cell. Conversely, when a getter element is activated, it removes a corresponding species from the vapor cell or vacuum cell. Getters may be used to eliminate contaminants to enhance gas purity and/or to counterbalance the gas output by a source to adjust a pressure or an amount of gas present within the cell.

Advantageously, by partitioning precursor material into physically separate elements that are independently activatable, the capacity of each element to generate or remove gas may be selected as a function of its physical dimensions, such as the area of the corresponding source or getter. This configuration enables incremental and predictable adjustment of gas conditions within the cell and supports improved control and repeatability relative to approaches that rely on bulk activation of a single precursor (e.g., AMDs).

While sources and getters may be activated via direct laser heating, the present embodiments provide improved thermal isolation relative to conventional approaches. For example, conventional laser-activation techniques, including direct laser writing applied to continuous (i.e., unpartitioned) precursor regions, often employ spatially diffuse beam profiles (e.g., Gaussian). Such profiles may produce non-uniform heating and, in turn, non-uniform activation of the precursor material, which may yield unwanted variability in the quantity of gas generated or removed.

To solve this problem, the present embodiments utilize precursor material partitioned into physically discrete elements (e.g., sources or getters), where “discrete” means the elements are physically separated and do not directly touch one another. In this configuration, each source and each getter constitutes an independently activatable element that is thermally isolated from adjacent elements such that activating any one element does not activate, and does not partially activate, any other element. This independence may be achieved by maintaining sufficient physical distance between elements to provide adequate thermal isolation. In other embodiments, where additional thermal decoupling is needed, pedestal structures may be used to further enhance thermal isolation. The pedestal's function and implementation are described in more detail below.

Advantageously, by tailoring the shapes and sizes of the physically discrete source and getter elements, the present embodiments enable fine-tuned control of gas pressure, purity, and quantity within the vapor cell or vacuum cell. Sequential activation of independently activatable elements of differing sizes provides incremental, predictable adjustments, enabling the system to converge on target conditions with precision. This approach overcomes limitations of previous technologies, such as AMDs, by delivering precise amounts of highly pure gas to vapor cells and vacuum cells.

The compatibility of the source and getter precursor materials with the processing conditions of the vapor cell or vacuum cell is important to maintaining functional integrity and performance, particularly because the sources and getters are partitioned into physically discrete structures intended to remain inactive until selectively activated. This compatibility encompasses fabrication and operational processes including evacuation, hermetic sealing, and thermal or chemical activation, so as to reduce the risk of premature activation, unintended cross-heating effects, or degradation that would impair the independent operation of the physically discrete sources and getters.

Integration of sources and getters into vapor cells may be executed through various means. In some embodiments, sources and getters are solid-state materials prepared separately from the vapor cell or vacuum cell and deposited onto designated substrates (e.g., by physical vapor deposition or chemical vapor deposition), after which post-deposition layers are precision patterned (e.g., by laser-based micromachining) into physically separated elements yielding sources or getters of selected shapes and sizes tailored to the specific needs of the vapor cell or vacuum cell at hand. The resulting gas generator may be mounted or affixed to the interior of the vapor cell or vacuum cell. For example, the gas generator may be mounted or affixed or disposed within an internal volume of a hermetically sealed microfabricated vapor cell or vacuum cell. Following evacuation and hermetic encapsulation of the vapor cell or vacuum cell, the gas generator may be operated by sequentially activating selected discrete source and getter elements to produce and/or remove controlled quantities of gas (e.g., to produce a precise amount of a highly pure gas).

Sources and getters may be arbitrarily shaped and sized based on what needs suit a specific design. There are many possible materials which may be used as sources of a gas of interest. These materials include, but are not limited to, bismuth(III) iodide, copper(I) iodide, and calcium-carbide with a solid-state source of water.

Similarly, there are many possible materials which may be used as getters for the gas of interest or unwanted species. The getter materials include, but are not limited to, aluminum (Al), gold (Au), silver (Ag), barium (Ba), barium oxide (BaO), bismuth (Bi), calcium (Ca), calcium oxide (CaO), cerium (Ce), cobalt (Co), chromium (Cr), copper (Cu), copper oxide (CuO), iron (Fe), iron(III) oxide (Fe₂O₃), hafnium (Hf), lanthanum (La), magnesium (Mg), magnesium oxide (MgO), manganese (Mn), manganese(II) oxide (MnO), molybdenum (Mo), niobium (Nb), nickel (Ni), palladium (Pd), platinum (Pt), scandium (Sc), tin (Sn), strontium (Sr), strontium oxide (SrO), tantalum (Ta), titanium (Ti), vanadium (V), tungsten (W), yttrium (Y), zinc (Zn), zinc oxide (ZnO), zirconium (Zr), or a combination thereof.

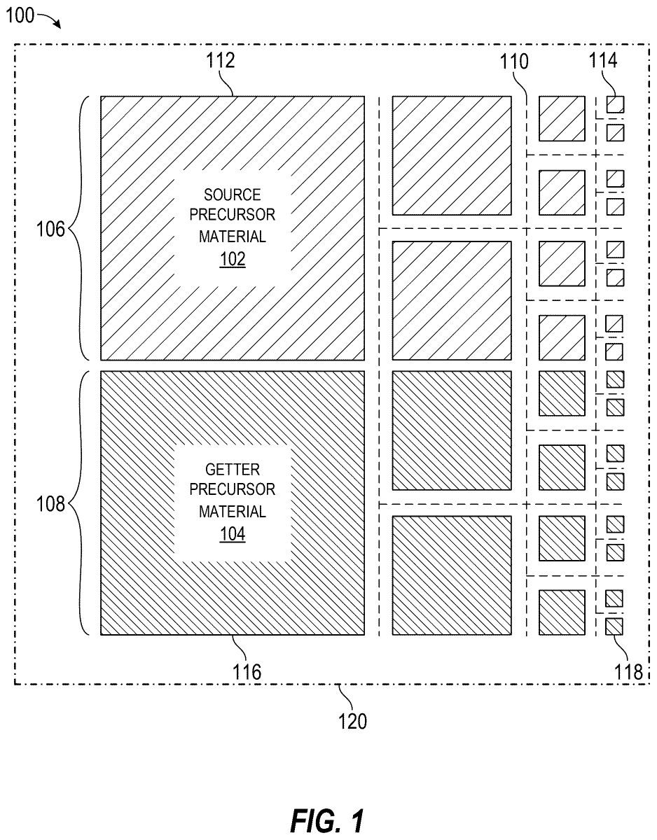

FIG. 1 shows a top view of a gas generator 100, in accordance with some of the present embodiments. The gas generator 100 includes a substrate 120 (outlined by the dash-dotted line) upon which a source precursor material 102 and a getter precursor material 104 are mounted. The source precursor material 102 is patterned into a plurality of sources 106, and the getter precursor material 104, different from the source precursor material 102, is patterned into a plurality of getters 108. Pattern lines (e.g., pattern line 110) are shown as dashed lines. In general, a precursor material (whether configured to function as a source or a getter) that is patterned into a plurality of discrete elements is referred to as a group of elements.

The plurality of sources 106 have different areas, including a first source 112 and a second source 114, such that the area of the first source 112 is larger than the area of the second source 114. Upon activation, each source of the plurality of sources 106 generates a gas quantity based on the area of the corresponding source. For example, activation of the first source 112 produces a greater quantity of gas compared to the second source 114, as the first source 112 possesses a larger surface area (and therefore contains more material of the source precursor material 102) than the second source 114. In some embodiments, all sources of the plurality of sources 106 have the same thickness.

Similarly, the plurality of getters 108 have different areas, including a first getter 116 and a second getter 118, such that the area of the first getter 116 is larger than the area of the second getter 118. Upon activation, each getter of the plurality of getters 108 removes a quantity of gas based on the area of the corresponding getter. For example, activation of the first getter 116 removes a greater quantity of gas compared to the second getter 118, as the first getter 116 possesses a larger surface area (and therefore contains more material of the getter precursor material 104) than the second getter 118. In some embodiments, all getters of the plurality of getters 108 have the same thickness.

The sources 106 and the getters 108 may vary in size according to any sort of scheme suitable for the intended application. For example, as illustrated in FIG. 1, the sources 106 are arranged in columns such that the sources in each successive column have an area that is one-fourth the area of the sources in the immediately preceding column. In other words, every source within a given column is sized so that its area is approximately 1/4 of the area of any source in the column before it.

Similarly, the getters 108 may be arranged in columns following the same geometric progression, enabling predictable scaling of gas generation and removal capacity. This arrangement facilitates hierarchical activation, where larger elements may be activated first to achieve coarse adjustments and progressively smaller elements may be activated to provide fine control over gas pressure and composition within the vapor cell or vacuum cell.

While FIG. 1 illustrates the sources 106 and the getters 108 patterned as square elements of varying size arranged within a rectangular perimeter, the present embodiments are not limited to this configuration. The sources 106 and the getters 108 may be patterned into any practical shape, including circles, rectangles, polygons, or irregular geometries, and may be distributed in any spatial arrangement suitable for the intended application. Likewise, the sizes of the sources 106 and the getters 108 may vary according to any scheme (such as geometric progression, uniform sizing, random variation, or hierarchical subdivision) and may be mixed within a single gas generator. This flexibility in shape, size, and distribution enables the design of gas generators tailored to diverse performance requirements without departing from the scope hereof.

In some embodiments, the gas generator 100 may be fabricated using, but not limited to, the following process. One or more precursor materials are deposited onto the substrate 120 using techniques such as physical vapor deposition, chemical vapor deposition, or other suitable deposition methods. After deposition, the source precursor material 102 is patterned into the plurality of sources 106, and the getter precursor material 104 is patterned into the plurality of getters 108. Patterning may be performed by laser trimming, machining with a dicing saw, or other precision fabrication techniques. The resulting configuration ensures that none of the plurality of sources 106 directly touch or contact each other, none of the plurality of getters 108 directly touch or contact each other, and none of the plurality of sources 106 directly touch or contact any of the plurality of getters 108. The completed gas generator 100 may then be mounted or affixed within a vacuum cell or vapor cell for subsequent activation.

In some of the embodiments, the source precursor material 102 includes, but is not limited to: (i) an iodine-generating precursor that, when activated, releases iodine gas, (ii) an acetylene-generating precursor that, when activated, releases acetylene gas, (iii) a rubidium-generating precursor that, when activated, releases rubidium gas, (iv) a cesium-generating precursor that, when activated, releases cesium gas, (v) a strontium-generating precursor that, when activated, releases strontium gas, (vi) a ytterbium-generating precursor that, when activated, releases ytterbium gas, (vii) a mercury-generating precursor that, when activated, releases mercury gas, (viii) an ammonia-generating precursor that, when activated, releases ammonia gas, (ix) a methane-generating precursor that, when activated, releases methane gas, or (x) a combination thereof.

Similarly, the getter precursor material 104 may include, but is not limited to, Al, Au, Ag, Ba, BaO, Bi, Ca, CaO, Ce, Co, Cr, Cu, CuO, Fe, Fe₂O₃, Hf, La, Mg, MgO, Mn, MnO, Mo, Nb, Ni, Pd, Pt, Sc, Sn, Sr, SrO, Ta, Ti, V, W, Y, Zn, ZnO, Zr, or a combination thereof. The getter precursor material 104, when activated, may remove: iodine gas, rubidium gas, cesium gas, ammonia gas, methane gas, acetylene gas, hydrogen gas, nitrogen gas, carbon monoxide, carbon dioxide, water vapor, oxygen, ozone, nitrous oxide, borides, borates, ethanol, methanol, isopropanol, organic solvents, organic acids, inorganic acids, or a combination thereof.

The choice of substrate material may be based on factors such as thermal conductivity, chemical compatibility with precursor materials, and mechanical stability under processing and operational conditions. In some of the embodiments, the substrate 120 has thermal properties that enhance thermal isolation of the plurality of sources 106 and the plurality of getters 108 such that activation of any one source or getter does not activate any other source or getter. For example, the substrate material may have a thermal conductivity less than 200 W/(m·K), 100 W/(m·K), 50 W/(m·K), 20 W/(m·K), 10 W/(m·K), 5 W/(m·K), 2 W/(m·K), or 1 W/(m·K).

Examples of the substrate material include, but are not limited to, aerogel, Al₂O₃, AlN, aluminosilicate glass, BN, borosilicate glass, CaF₂, carbon nanotubes, cordierite, diamond, GaAs, GaN, Ge, silica (e.g., silica gel), glass, graphite, InP, liquid crystal polymer, LiNbO₃, LiTaO₃, low temperature co-fired ceramic, MgF₂, MgO, parylene, PEEK, photosensitive glass, polyimide, porous silicon, PTFE, silicon, silicon carbide, silicon nitride, SiO₂, SiₓGe_{y}, xerogel, YAG, ultra-low expansion glass, ZnS, ZnSe, ZrO₂, or a combination thereof.

The substrate 120 may be shaped or sized to lower thermal conductance between adjacent elements (e.g., sources and getters), thereby enhancing thermal isolation. For example, the substrate 120 may be made thin to lower the effective cross-sectional area available for heat transfer. This adjustment increases thermal resistance along the path through which heat would otherwise conduct between adjacent elements (i.e., the thermal conduction path through the substrate 120). A thinner substrate may limit lateral heat spreading and confine thermal energy to the activated element, reducing the likelihood of unintended activation of neighboring sources or getters.

Alternatively or additionally, the substrate 120 may be made large to increase the physical distance between the elements (e.g., sources and getters) mounted on the substrate 120. Greater spacing between sources and getters lengthens the thermal conduction path and further improves thermal isolation. In some embodiments, the substrate 120 may include regions of varying thickness or stepped profiles to locally tailor thermal resistance. For example, the substrate 120 may incorporate recesses, trenches, or cutouts between adjacent elements to reduce the effective thermal cross-section and create localized thermal barriers. These geometric modifications may be combined with material selection and pedestal structures to achieve a level of thermal isolation suitable for precise and independent activation of each source and getter.

In some of the embodiments, the spatial arrangement of the plurality of sources 106 and the plurality of getters 108 on the substrate 120 is configured to maintain a minimum separation distance between adjacent elements (i.e., sources and getters) to promote thermal isolation and reduce the likelihood of unintended activation of neighboring elements during activation (e.g., localized heating). For example, the plurality of sources 106 may be arranged so that the minimum distance between any two sources is not less than 0.005 mm, 0.01 mm, 0.05 mm, 0.1 mm, 0.5 mm, 1 mm, or 5 mm. Similarly, the plurality of getters 108 may be arranged so that the minimum distance between any two getters is not less than 0.005 mm, 0.01 mm, 0.05 mm, 0.1 mm, 0.5 mm, 1 mm, or 5 mm. In addition, the plurality of sources 106 and the plurality of getters 108 may be arranged so that the minimum distance between any one source and any one getter is not less than 0.005 mm, 0.01 mm, 0.05 mm, 0.1 mm, 0.5 mm, 1 mm, or 5 mm. These spacing parameters may be selected and adjusted based on factors such as the thermal conductivity of the substrate 120, the sizes of the sources 106 and the getters 108, and the activation method to support reliable and independent operation of each source and getter.

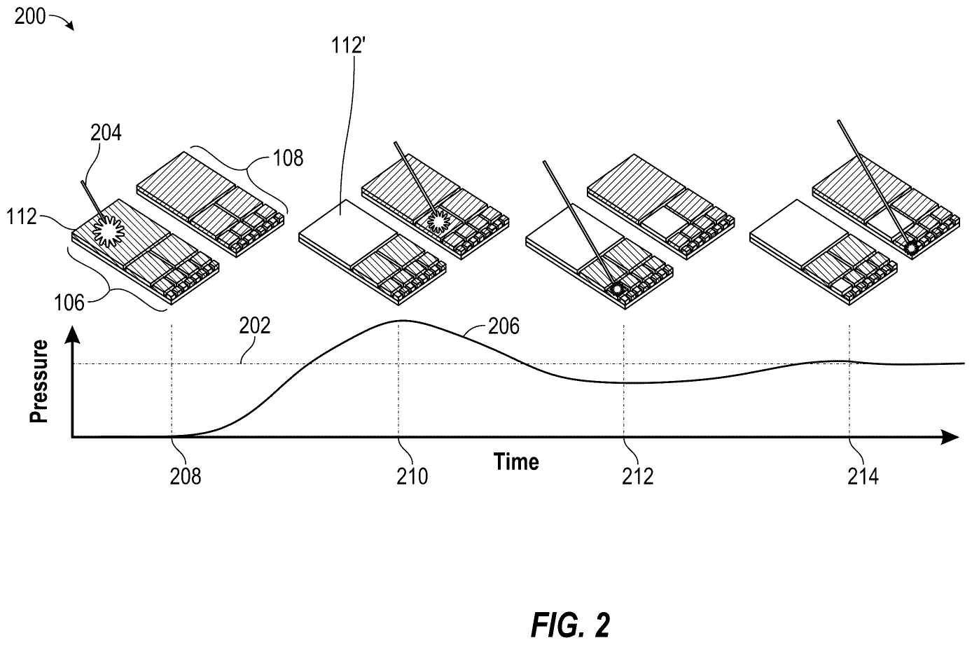

FIG. 2 illustrates one example of a method 200 for generating a gas within a vacuum cell or a vapor cell by sequentially activating the gas generator 100 of FIG. 1. In this embodiment, sources and getters of different sizes are selectively activated by a laser 204 to adjust a gas pressure 206 within the vacuum cell or vapor cell toward a target pressure 202. For clarity, the laser 204 is labeled only once in FIG. 2. This sequential activation is depicted as a time series over four consecutive steps.

The plurality of sources 106 and the plurality of getters 108 are patterned into discrete elements of practical shapes and sizes, such that the quantity of gas released or removed upon activation is determined by the physical dimensions (e.g., area) and density of each element. Activating progressively smaller sources and getters enables finer adjustments to the gas pressure 206. Sequential laser activation of these sources and getters may be part of a feedback loop to achieve the target pressure 202 within an evacuated and hermetically sealed vapor cell or vacuum cell. Although FIG. 2 depicts the sources 106 formed from the source precursor material 102 (illustrated with one striped pattern) and the getters 108 formed from the getter precursor material 104 (illustrated with a different striped pattern orientation), the present embodiments include sources and getters composed of any number of one or more distinct precursor materials.

The sources 106 and the getters 108 of the gas generator 100 are arranged to enhance and maintain thermal isolation, such that activation of any one source (of the plurality of sources 106) or any one getter (of the plurality of getters 108) does not activate or partially activate any other source or getter (of the plurality of sources 106 and the plurality of getters 108, respectively). This independence enables precise control of gas generation and removal within the vacuum cell or vapor cell. In the example shown in FIG. 2, the sources 106 and the getters 108 are patterned as square elements of varying size distributed within a rectangular perimeter, with sizes decreasing according to a geometric sequence. However, the present embodiments are not limited to this configuration. The sources 106 and the getters 108 may be patterned into any shape, arranged in any spatial distribution, and sized according to any scheme, whether geometric, uniform, random, hierarchical, or otherwise. This flexibility in shape, size, and distribution enables the design of gas generators tailored to diverse performance requirements while maintaining the ability to activate each source or getter independently.

In the example shown in FIG. 2, the method 200 begins at an initial time 208 when the laser 204 activates the first source 112 within the vacuum cell or vapor cell, generating an amount of gas based on (e.g., proportional to) the area of the first source 112 and establishing an initial gas pressure. In this step, the first source 112 is fully activated, and the resulting fully activated first source (shown as a blank element and labeled 112′) thereafter does not produce additional gas upon further activation. Advantageously, the sources 106 and the getters 108 are sufficiently thermally isolated so that activation of the first source 112 does not activate any other source or getter.

In this example, the initial activation causes the gas pressure 206 to exceed the target pressure 202. To reduce the gas pressure 206, a suitably sized one of the getters 108 is activated at a subsequent time 210 to remove a proportional amount of gas, bringing the gas pressure 206 closer to, but now slightly below, the target pressure 202. At a time 212, a second one of the sources 106 is activated, raising the gas pressure 206 slightly above the target pressure 202. Finally, at a time 214, a second one of the getters 108 is activated to lower the gas pressure 206 closer to the target pressure 202. This example of the method 200 demonstrates how activating progressively smaller sources and getters enables precise and repeatable adjustments to the gas pressure 206 within the vacuum cell or vapor cell.

In some embodiments, the gas generator 100 of FIG. 1 may be integrated with an automated control system to regulate gas conditions within a hermetically sealed container, such as a vacuum cell or vapor cell. The control system includes a controller operatively coupled to one or more sensors and to an energy source. The one or more sensors are configured to measure one or more gas parameters such as a pressure, a composition, and/or a purity, and may include optical absorption spectrometers, cavity ring-down spectrometers, pressure transducers, residual gas analyzers, photodetectors, or combinations thereof. The energy source, such as a laser, resistive heater, or other directed heating element, is configured to selectively activate individual sources of the plurality of sources 106 and individual getters of the plurality of getters 108 under the direction of the controller.

In some embodiments, the controller executes a feedback loop that iteratively selects which sources or getters to activate based on real-time measurements of a gas within a vapor cell or vacuum cell obtained from one or more sensors. Upon detecting a deviation of a measured gas parameter (such as pressure or composition) from a target condition, the controller identifies an appropriately sized source or getter from the plurality of sources 106 and getters 108 to counteract the deviation. The selected element is then activated to generate or remove a predictable quantity of gas, thereby adjusting the measured gas parameter toward the target condition. In some embodiments, larger sources or getters may be activated first to provide coarse adjustments, followed by progressively smaller elements for fine control, enabling incremental convergence toward the target condition with high precision. The feedback loop may continue until the measured gas parameter falls within a predetermined tolerance of the target condition or until all available sources or getters have been activated.

In some embodiments, the feedback loop employs spectroscopic sensing to determine the concentration of a particular gas species within the vapor cell or vacuum cell. Based on this measurement, the controller may selectively activate getters to remove specific contaminants or activate sources to introduce the intended gas species. This closed-loop trimming approach enables adaptive, repeatable, and automated regulation of gas pressure and composition, supporting applications that require stringent environmental stability, such as atomic clocks, laser stabilization systems, and other precision devices.

In some embodiments, the discrete element sizes available for selection by the controller are provided by subdividing a precursor material into physically discrete, independently activatable elements arranged in a hierarchical manner. This hierarchical subdivision creates a range of element sizes that correspond to different quantities of gas generation or removal. In such embodiments, the available element sizes define discrete adjustment steps for changing a monitored parameter, such as gas pressure or composition. For example, a controller or operator may iteratively (i) measure a deviation between the monitored parameter and a target condition, (ii) select a source or getter based on whether the deviation indicates that the monitored parameter should be increased or decreased, and (iii) select an element size based on the magnitude of the deviation so as to reduce the deviation in successive activations.

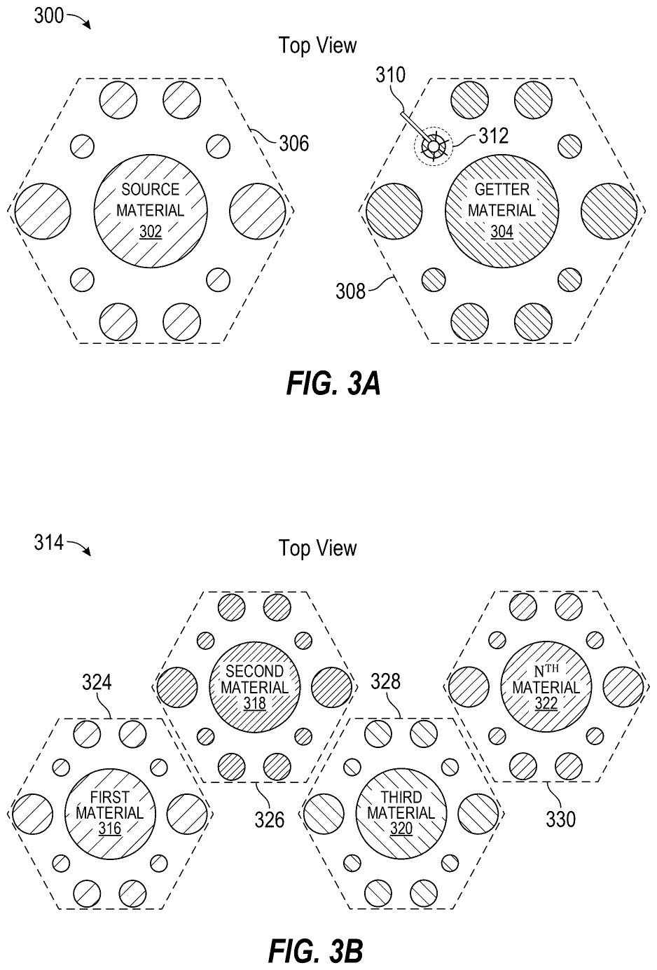

FIG. 3A shows a top view of a gas generator 300 that includes a source precursor material 302 patterned into circular sources of different sizes, thereby forming a plurality of sources 306 arranged in a hexagon. Similarly, a getter precursor material 304 is patterned into circular getters of different sizes to form a plurality of getters 308 arranged in a hexagon. Advantageously, circular sources and getters may be best suited for materials that diffuse heat radially, making it easier to size sources and getters while maintaining thermal isolation. A heating laser 310 activating one of the getters 308 illustrates a spatial extent of heating 312, indicating that thermal isolation is maintained for such a system.

FIG. 3B shows a top view of a gas generator 314 that is similar to the gas generator 300 of FIG. 3A. The gas generator 314 includes four groups of elements, each formed of a distinct precursor material: a first group of elements 324 formed of a first material 316, a second group of elements 326 formed of a second material 318, a third group of elements 328 formed of a third material 320, and an Nth group of elements 330 formed of an Nth material 322. Each group of elements is formed from a distinct precursor material (e.g., a sheet of precursor material) that is laser-trimmed to create the respective group of elements. Each distinct precursor material is patterned into either a plurality of sources or a plurality of getters. Furthermore, the gas generator 314 may be optimized to fit a given number of groups of elements on a given surface by patterning each distinct precursor material into sources or getters such that the elements within each group form an arrangement (such as a triangle, a rectangle, or a hexagon) that efficiently organizes that group to satisfy design requirements.

While FIGS. 1, 2, 3A, and 3B show the plurality of sources 106 and the plurality of getters 108 shaped as squares and circles, the sources 106 and the getters 108 may have any shape without departing from the scope hereof (e.g., rectangles, ovals, pentagons, irregular polygons). While FIGS. 1, 2, 3A, and 3B show all elements (e.g., sources or getters) within the same group as having the same shape, elements within the same group may have different shapes without departing from the scope hereof. While FIGS. 1, 2, 3A, and 3B show embodiments where the number of the sources 106 is the same as the number of the getters 108, the present embodiments also include gas generators in which the number of the sources 106 is different from the number of the getters 108.

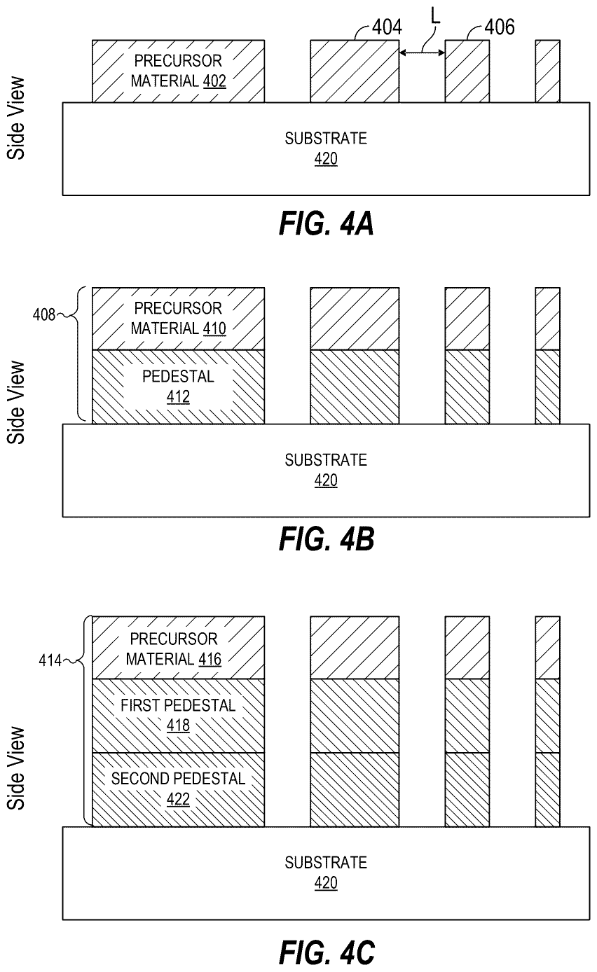

FIG. 4A shows a side view of a patterned precursor material 402 (i.e., a plurality of sources or a plurality of getters), in accordance with some of the present embodiments. The patterned precursor material 402 is partitioned into discrete elements (e.g., sources or getters) of various sizes and bonded directly onto a substrate 420. The elements are thermally isolated from one another if activation of any one element does not activate, or partially activate, any other element. Such thermal isolation may be adequate provided that the thermal conductivity of the substrate 420 is low enough, elements are spaced far enough apart from each other, and the effective cross-sectional area for heat flow between adjacent elements is small enough. For example, in a conductive heat-transfer model, thermal resistance between adjacent elements through an intervening material (e.g., the substrate 420) scales with separation distance L and inversely with both material thermal conductivity k and cross-sectional area A, for example as Rₜₕ≈L/(k·A). To illustrate this concept, FIG. 4A shows an element 404 spaced a distance L from an adjacent element 406.

FIG. 4B shows a side view of a pedestaled precursor structure 408, in accordance with some of the present embodiments. The pedestaled precursor structure 408 includes a patterned precursor material 410 (i.e., a plurality of sources or a plurality of getters) and a pedestal 412. When the thermal conductivity of the substrate 420 is too high to ensure thermal isolation between sources and getters, an additional layer may be included to separate the patterned precursor material 410 from the substrate 420. Thermal isolation may be attained by including the pedestal 412 sandwiched between the substrate 420 and the patterned precursor material 410. The pedestal 412 is made of a material with the requisite thermal properties to ensure thermal isolation between adjacent elements. The pedestaled precursor structure 408 may be fabricated by depositing a first layer (e.g., a layer of material forming the pedestal 412) onto a substrate and then a second layer (e.g., a layer of precursor material forming the patterned precursor material 410) on top of the first layer. The resulting structure may then be patterned into discrete elements (e.g., via laser-trimming).

FIG. 4C illustrates a side view of a pedestaled precursor heterostructure 414, in accordance with some of the present embodiments, that is similar to the pedestaled precursor structure 408 of FIG. 4B but features a heterostructure of layers that form a pedestal. The pedestaled precursor heterostructure 414 includes a patterned precursor material 416 bonded to a first pedestal 418, which is itself bonded to a second pedestal 422. The second pedestal 422 is in turn bonded to the substrate 420. It is advantageous in certain circumstances to layer a precursor material (e.g., the patterned precursor material 416) atop a pedestal with high thermal conductivity (e.g., the first pedestal 418), which itself is positioned on a pedestal with low thermal conductivity (e.g., the second pedestal 422). This configuration promotes uniform heat distribution throughout an activated source or getter, and limits thermal diffusion into other sources or getters (see, for example, the spatial extent of heating 312 in FIG. 3A). Such a design permits the full activation of one source or getter without activating, or partially activating, any other source or getter. Additionally, the pedestals and sources or getters allow for thermal isolation in the form of an air or vacuum gap between the pedestals.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A gas generator, comprising:
a plurality of sources composed of a first material, the plurality of sources including a first source having a first source area and a second source having a second source area less than the first source area;
a plurality of getters composed of a second material different from the first material, the plurality of getters including a first getter having a first getter area and a second getter having a second getter area less than the first getter area; and
a substrate on which the plurality of sources and the plurality of getters are mounted such that none of the plurality of sources directly touch each other, none of the plurality of getters directly touch each other, and none of the plurality of sources directly touch the plurality of getters.

2. The gas generator of claim 1, wherein:
the plurality of sources further comprises a third source having a third source area less than the second source area; and
the plurality of getters further comprises a third getter having a third getter area less than the second getter area.

3. The gas generator of claim 1, further comprising a second plurality of sources composed of a third material different from the first material and different from the second material.

4. The gas generator of claim 1, further comprising a second plurality of getters composed of a fourth material different from the first material and different from the second material.

5. The gas generator of claim 1, wherein:
the second source area is one-half the first source area;
a third source of the plurality of sources has a third source area one-fourth the first source area;
the second getter area is one-half the first getter area; and
a third getter of the plurality of getters has a third getter area one-fourth the first getter area.

6. The gas generator of claim 1, wherein:
the plurality of sources comprises:
a third source and a fourth source, each of the third source and the fourth source having an area one-fourth the first source area; and
a fifth source and a sixth source, each of the fifth source and the sixth source having an area one-sixteenth the first source area; and
the plurality of getters comprises:
a third getter and a fourth getter, each of the third getter and the fourth getter having an area one-fourth the first getter area; and
a fifth getter and a sixth getter, each of the fifth getter and the sixth getter having an area one-sixteenth the first getter area.

7. The gas generator of claim 1, wherein the first material comprises:
an iodine-generating precursor that, when activated, releases iodine gas;
an acetylene-generating precursor that, when activated, releases acetylene gas;
a rubidium-generating precursor that, when activated, releases rubidium gas;
a cesium-generating precursor that, when activated, releases cesium gas;
a strontium-generating precursor that, when activated, releases strontium gas;
a ytterbium-generating precursor that, when activated, releases ytterbium gas;
a mercury-generating precursor that, when activated, releases mercury gas;
an ammonia-generating precursor that, when activated, releases ammonia gas; or
a methane-generating precursor that, when activated, releases methane gas.

8. The gas generator of claim 1, wherein the second material, when activated, removes iodine (I₂) gas, rubidium (Rb) gas, cesium (Cs) gas, ammonia gas, methane gas, acetylene gas, hydrogen (H₂) gas, nitrogen (N₂) gas, carbon monoxide, carbon dioxide, water vapor, oxygen, ozone, nitrous oxide, borides, borates, ethanol, methanol, isopropanol, organic solvents, organic acids, inorganic acids, or a combination thereof.

9. The gas generator of claim 1, wherein the second material comprises Al, Au, Ag, Ba, BaO, Bi, Ca, CaO, Ce, Co, Cr, Cu, CuO, Fe, Fe₂O₃, Hf, La, Mg, MgO, Mn, MnO, Mo, Nb, Ni, Pd, Pt, Sc, Sn, Sr, SrO, Ta, Ti, V, W, Y, Zn, ZnO, Zr, or a combination thereof.

10. The gas generator of claim 1, wherein the substrate has a thermal conductivity of less than 200 W/(m·K).

11. The gas generator of claim 1, wherein the substrate is composed of aerogel, Al₂O₃, AlN, aluminosilicate glass, BN, borosilicate glass, CaF₂, carbon nanotubes, cordierite, diamond, GaAs, GaN, Ge, gelled silica, glass, graphite, InP, liquid crystal polymer, LiNbO₃, LiTaO₃, low temperature co-fired ceramic, MgF2, MgO, parylene, PEEK, photosensitive glass, polyimide, porous silicon, PTFE, silicon, silicon carbide, silicon nitride, SiO₂, SiₓGe_{y}, xerogel, YAG, ultra-low expansion glass, ZnS, ZnSe, ZrO₂, or a combination thereof.

12. The gas generator of claim 1, wherein:
the plurality of sources are not closer than 0.01-1 mm from each other;
the plurality of getters are not closer than 0.01-1 mm from each other; and
the plurality of sources are not closer than 0.01-1 mm from the plurality of getters.

13. A hermetically sealed microfabricated cell assembly, comprising:
a hermetically sealed microfabricated vapor cell or vacuum cell; and
the gas generator of claim 1 disposed within an internal volume of the hermetically sealed microfabricated vapor cell or vacuum cell.

14. A method for generating a gas within a vacuum cell, comprising:
activating a first source of a plurality of sources located within the vacuum cell, the plurality of sources being composed of a first material, the first source having a first source area, wherein the first source, when activated, releases the gas from the first source into the vacuum cell having an initial pressure based on the first source area;
activating a first getter of a plurality of getters located within the vacuum cell, the plurality of getters being composed of a second material different from the first material, the first getter having a first getter area, wherein the first getter, when activated, removes a portion of the gas based on the first getter area such that the gas has a subsequent pressure that is less than the initial pressure; and
activating a second source of the plurality of sources, the second source having a second source area less than the first source area, wherein said activating the second source releases, from the second source and adding to the gas in the vacuum cell, an additional gas based on the second source area such that the gas has a second subsequent pressure that is higher than the subsequent pressure.

15. The method of claim 14, wherein:
said activating the first source does not activate any other source of the plurality of sources;
said activating the first getter does not activate any other getter of the plurality of getters;
and said activating the second source does not activate any other source of the plurality of sources.

16. The method of claim 14, further comprising activating a second getter from the plurality of getters, the second getter having a second getter area less than the first getter area, wherein said activating the second getter removes, from the gas in the vacuum cell, an additional portion of the gas based on the second getter area such that the gas has a third subsequent pressure that is less than the second subsequent pressure.

17. The method of claim 16, wherein said activating the second getter of the plurality of getters does not activate any other getter of the plurality of getters.

18. A method for fabricating the plurality of sources and the plurality of getters of the gas generator of claim 1, comprising:
patterning a post-deposition layer composed of the first material to create the plurality of sources; and
patterning an additional post-deposition layer composed of the second material to create the plurality of getters.

19. The method of claim 18, further comprising mounting or affixing the gas generator within a vacuum cell.

20. The method of claim 18, wherein:
said patterning the post-deposition layer includes laser trimming the post-deposition layer
or machining, with a dicing saw, the post-deposition layer; and
said patterning the additional post-deposition layer includes laser trimming the post-deposition layer or machining, with a dicing saw, the post-deposition layer.
